(19) United States
(12) Patent Application Publication
(10) Pub. No.: US 2024/0245490 A1
(43) Pub. Date: Jul. 25, 2024

(54) METHOD FOR DISTRIBUTING ORTHODONTIC ALIGNERS

(71) Applicant: DENTAL MONITORING, Paris (FR)

(72) Inventor: Thomas PELLISSARD, Maisons-Alfort (FR)

(21) Appl. No.: 18/562,155

(22) PCT Filed: May 24, 2022
(86) PCT No.: PCT/EP2022/064123

§ 371 Date: Nov. 17, 2023

(30) Foreign Application Priority Data
May 25, 2021 (FR) 2105399

(51) Int. Cl. A61C 7/00; A61B 1/24; A61B 1/32; A61C 7/08; A61C 13/34; G16H 20/40

(52) U.S. Cl. CPC A61C 7/002; A61B 1/24; A61B 1/32; A61C 7/08; A61C 13/34; G16H 20/40

(57) ABSTRACT

A method for distributing orthodontic aligners intended for an orthodontic treatment of a user's dental arch. The orthodontic treatment is defined by an initial orthodontic treatment plan. At a current instant during an “anterior” part of the orthodontic treatment, acquiring, on the part of the user and using a cellphone, a current representation of the dental arch. Then, updating the initial orthodontic treatment plan on the basis of the current representation, and defining, on the basis of the updated initial orthodontic treatment plan, a “posterior” part of the initial orthodontic treatment following the anterior part of the orthodontic treatment. Then, designing and manufacturing only the orthodontic aligners necessary for the “posterior” part, referred to as “posterior orthodontic aligners,” the number of posterior orthodontic aligners being more than or equal to 1 and fewer than 20. Presenting the one or more posterior orthodontic aligners to the user.

### TECHNICAL FIELD

The present invention relates to a method for distributing orthodontic aligners.

The invention also relates to a computer system for implementing such a method.

### PRIOR ART

An orthodontic aligner conventionally takes the form of a removable, one-piece appliance, conventionally made of a transparent polymer material, that is shaped to follow the successive teeth of the arch to which it is attached. It comprises a tray, with the overall shape of a “U”, that is shaped so that multiple teeth of an arch, generally all of the teeth of an arch, may be accommodated therein.

The shape of the tray is determined to ensure the attachment of the aligner to the teeth, but also depending on a desired target positioning for the teeth. More specifically, the shape is determined such that, when the aligner is in its use position, it exerts stresses that tend to displace the treated teeth toward the target positioning.

Conventionally, at the start of the orthodontic treatment, the shapes that the various aligners must take at various instants during the treatment are determined, then all of the corresponding aligners are manufactured. To this end, it is known to carry out the following steps:
generating, at an initial instant t₁, typically at the start of the treatment, a three-dimensional digital model, referred to as “initial model”, of a dental arch of the user, said arch being in an initial configuration, and segmenting the initial model into tooth models;
determining an orthodontic treatment suitable for modifying the arch from said initial configuration, via intermediate configurations at respective intermediate instants tₙ, with n being between 1 and N, to a final configuration, at a final instant t_{N+1};
deforming the initial model so as to generate intermediate and final models representing the dental arch in the intermediate and final configurations, respectively;
determining, on the basis of the initial, intermediate and final models, a series of N aligners, the first aligner being intended to be worn until the instant t₂ and the nth aligner being intended to be worn from the instant tₙ to the instant tₙ₊₁;
manufacturing at least some of the aligners.

All of the manufactured aligners are then provided to the user in order that, at the predetermined intermediate instants, they may change the aligner.

At regular intervals during the treatment, the user goes to the orthodontist for a visual check-up, notably to verify whether the displacement of the teeth is as expected and whether the aligner being worn is still suitable for the treatment.

In particular, the orthodontist may visually diagnose unseating of the aligner. Specifically, the bottom of the tray has a substantially complementary shape to that of the free ends of the teeth. Consequently, the contour of the bottom of the tray can be compared to the contour of the teeth in order to evaluate a gap between the bottom of the tray and one or more free ends of the teeth.

If unseating is detected, the orthodontist takes a new impression of the teeth, or, equivalently, a new scan of the teeth, and then repeats the process described above in order to design and manufacture a new series of aligners.

It is stressful for the user to have to go to the orthodontist. The user's confidence in their orthodontist may also be undermined. Finally, it results in an additional cost. The number of check-ups at the orthodontist must therefore be limited.

Moreover, a poorly fitted aligner may also be unsightly.

To solve these problems, the Applicant has proposed, in EP 3 412 245, a method for evaluating the shape of an orthodontic aligner worn by a user.

This method advantageously makes it possible to remotely detect unseating of the aligner. It considerably facilitates evaluation of the suitability of the aligner for the treatment. In particular, it may be implemented on the basis of simple images, and in particular on the basis of photographs or films, that can be taken without special precautions, for example by the user. The number of appointments with the orthodontist can therefore be limited.

The Applicant has also proposed, in FR 3 096 255, a method for manufacturing orthodontic aligners by means of a method for generating a digital model of a dental arch by deformation of an intermediate model.

This method advantageously makes it possible to avoid the user performing a new scan for the generation of new trays. This is because said method makes it possible, on the basis of simple images, and in particular on the basis of photographs or films, that can be taken without special precautions, for example by the user, to obtain a current model from an intermediate model, and thus to design and then manufacture new aligners.

The methods described above are based on rigorous participation of the user in the orthodontic treatment. However, for various reasons, the user might be distracted and not rigorously comply with the orthodontic treatment.

There is therefore a need to improve the user's compliance with the orthodontic treatment while still preserving the advantages afforded by the methods described above.

Furthermore, there is a permanent need to shorten the duration of the orthodontic treatment.

Lastly, there is a need to limit the impact of the orthodontic treatment on the environment.

One aim of the invention is to at least partially meet these needs.

### PRESENTATION OF THE INVENTION

### Summary of the Invention

The invention provides a method for distributing orthodontic aligners intended for an orthodontic treatment of a user's dental arch, said method comprising a cycle of the following steps:
A) at a current instant during an “anterior” part of the orthodontic treatment, acquiring, on the part of the user and using a cellphone, a current representation of said dental arch, then
B) optionally, using the current representation, determining a digital three-dimensional model representing the dental arch at the current instant, referred to as “current” model, and then
C) taking the current representation, preferably the current model determined in step B), as a basis, more preferably by comparing the current model and a “target” digital three-dimensional model of the dental arch, to design and manufacture only the orthodontic aligners necessary for a “posterior” part of the orthodontic treatment following said anterior part of the orthodontic treatment, referred to as “posterior orthodontic aligners”, and presenting the one or more posterior orthodontic aligners to the user.

As will be seen in more detail in the rest of the description, such a method advantageously makes it possible to meet the needs expressed above. Specifically, the inventors have observed that the orthodontic aligners are all the more effective if they are used very shortly after the treatment plan is produced. The reality of the orthodontic treatment may then deviate from the treatment plan, such that the orthodontic aligners used are less and less suited to the reality of the user's dental situation. By designing and manufacturing the orthodontic aligners throughout the orthodontic treatment, it is thus possible to provide orthodontic aligners that are still well suited to reality, and therefore to shorten the duration of the treatment.

Furthermore, in step A), the user actively participates in their orthodontic treatment, thereby improving compliance.

Lastly, conventionally, all the orthodontic aligners necessary for the orthodontic treatment are manufactured at the same time and presented to the user. However, it is rare that the orthodontic treatment progresses exactly as initially intended and many orthodontic aligners are ultimately not worn by the user. According to the invention, in step C), only the orthodontic aligners necessary for the posterior part of the orthodontic treatment are manufactured, thereby avoiding the manufacture and transport of orthodontic aligners that will not be used. The environmental impact of the orthodontic treatment is advantageously reduced as a result.

The target model is preferably a model representing the dental arch in a configuration aimed at for the end of the posterior part of the orthodontic treatment or for the end of the orthodontic treatment.

In one embodiment, the posterior part may be a post-orthodontic treatment period, the one or more orthodontic aligners then preferably being one or more support appliances.

Preferably, before the current instant, a notification is sent to said cellphone to provide a reminder of the need to acquire said current representation. This improves compliance with the orthodontic treatment.

Sending a notification may depend on a number of observations of a poor fit of the orthodontic aligner that were made beforehand in the implementation of the orthodontic treatment.

For example, the sending of the notification can be triggered, preferably automatically, if it is observed multiple times in succession that the orthodontic aligner worn by the user is not suited to the orthodontic treatment, for example owing to unseating.

The notification can be in particular be sent at any instant during a posterior part defined during a cycle of steps A), B), C) according to the invention that was implemented beforehand.

Preferably, in step A), the current representation is acquired extraorally.

Preferably,
a dental retractor or
a mouthpiece to which the cellphone is attached
is used by the user in step A).

Preferably, in step A), the current instant is before the instant intended for the end of the anterior part of the orthodontic treatment, or “anterior final instant”. The temporal interval between the current instant and the first final instant is preferably longer than 1 day, 2 days, 4 days, 5 days, 1 week and/or preferably shorter than 1 month, preferably shorter than 4 weeks, preferably shorter than 3 weeks, preferably shorter than 2 weeks. Advantageously, the cycle can be performed quickly enough for the user to receive the posterior orthodontic aligners before the anterior final instant, thereby avoiding interruption of the orthodontic treatment. However, the current instant is close to the anterior final instant, such that the current model correctly represents the configuration of the dental arch at the anterior final instant.

The current instant may also be the instant intended for the end of the anterior part of the orthodontic treatment.

Preferably, in step A), the current representation is made up of a model acquired by a scanner integrated in the cellphone and/or of one or more images acquired by a camera integrated in the cellphone, and at least one, preferably each image preferably is a photo or an image extracted from a film.

Preferably, in step B), the current model is made up of said acquired model or is generated from the current images and/or the acquired model.

In step B), the current model may be in particular generated
by means of a portable scanner integrated in the cellphone, or
by means of a neural network from one or more current images acquired using the cellphone, or
by assembling, preferably by means of a neural network, “historical” tooth models as a function of a model acquired using a portable scanner integrated in the cellphone and/or one or more current images acquired using the cellphone, or
by deforming, by means of a metaheuristic method, a model of the dental arch generated before the current instant, preferably before the start of the orthodontic treatment, for example the model of the dental arch used to design the orthodontic aligner worn by the user at the current instant, such that the deformed model best fits the one or more current images.

These methods advantageously make it possible to generate the current model remotely, that is to say without the user needing to visit a dental healthcare professional, in particular an orthodontist.

The neural network may in particular be trained by providing it with dental arch images as input and models corresponding to these images as output, with a model corresponding to an image when it is possible, by observing it, to obtain a view of the model that can be superposed in alignment with the image.

Preferably, at the start of step C), with the current model, the treatment plan used to define the orthodontic treatment is updated so as to define said posterior part of the orthodontic treatment. The orthodontic treatment is thus adapted to take into account the real configuration of the dental arch at the current instant, modeled using the current model. The updating consists in defining a new orthodontic treatment plan designed to lead, from the current instant, to the configuration of teeth according to the target model defined for the end of the orthodontic treatment. The posterior part is therefore at least one part of this new orthodontic treatment plan. Preferably, in step C), said posterior part of the orthodontic treatment is defined such that the number of posterior orthodontic aligners is between 1 and 15. Advantageously, this considerably improves the effectiveness of the orthodontic treatment. To limit the number of times orthodontic aligners are sent to the user, it is useful to send them multiple orthodontic aligners, or even all the orthodontic aligners necessary for the orthodontic treatment, as soon as the orthodontic treatment starts.

Furthermore, to limit the amount of orthodontic aligners manufactured, it is useful to define the posterior part of the orthodontic treatment such that it corrects a deviation from the initially defined orthodontic treatment, or “initial orthodontic treatment”.

The deviation can be determined by measuring a difference between the position of the teeth at the current instant and a theoretical position of said teeth in accordance with the orthodontic treatment plan, for example by comparing a current model with an intermediate model anticipating the position of the teeth substantially for the current instant, for example produced at the start of the orthodontic treatment.

Said posterior part of the orthodontic treatment is then defined such that, during said posterior part of the orthodontic treatment, said teeth return to a position in accordance with said initial orthodontic treatment plan. In particular, the deviation may concern only some teeth, and in particular teeth which shift more slowly than others, with it being possible for the posterior part to be used to correct this retarded movement of these teeth without modifying the positioning of the other teeth.

At the end of the posterior part of the orthodontic treatment, it is thus advantageously possible to return to a treatment in accordance with the initial orthodontic treatment. Preferably, only the orthodontic aligners necessary for this correction of the orthodontic treatment are manufactured.

To correct a deviation from an initial orthodontic treatment, the posterior part of the orthodontic treatment is preferably defined such that the number of posterior orthodontic aligners is fewer than 30, preferably fewer than 15, preferably fewer than 10, preferably fewer than 5, preferably fewer than or equal to 3.

In particular to correct a deviation from an initial orthodontic treatment, the number of posterior orthodontic aligners is preferably determined as a function of the duration of the anterior part of the orthodontic treatment, preferably is proportional to the duration of the anterior part of the orthodontic treatment.

In particular to correct a deviation from an initial orthodontic treatment, the orthodontic treatment may be the initial orthodontic treatment of which a segment
that ends before the end of the initial orthodontic treatment has been replaced with said posterior part, it advantageously being possible for the orthodontic aligners designed and manufactured for the period of the initial orthodontic treatment to be used after said posterior part, or
that ends with the initial orthodontic treatment has been replaced with said posterior part.

A “corrective orthodontic aligner” refers to a posterior orthodontic aligner which has not been defined by the initial orthodontic treatment plan and is intended to correct a deviation. All of the posterior orthodontic aligners or some of the posterior orthodontic aligners may be corrective orthodontic aligners.

In one embodiment, to correct a deviation from an initial orthodontic treatment, the posterior part of the orthodontic treatment defines only corrective orthodontic aligners and is inserted into the initial orthodontic treatment plan without modifying it, except to temporally offset it. In other words, the orthodontic aligners manufactured according to the initial orthodontic treatment plan all remain usable, with the posterior part of the orthodontic treatment only correcting the deviation.

This embodiment is advantageous in that no initial orthodontic aligner is lost. However, it requires rapid detection of a deviation in order for the correction to be possible. A cycle of steps A) to C) is preferably implemented after the user has used a given number of orthodontic aligners, preferably after they have used fewer than 4, preferably fewer than 3, preferably fewer than 2 orthodontic aligners.

For example, the user's dental situation can be monitored every month, or after every use of two or three orthodontic aligners. Any deviation is then limited and a return to the initial orthodontic treatment plan is then possible with a reduced number of corrective orthodontic aligners. Preferably, at least in this embodiment, the number of corrective orthodontic aligners is fewer than 4, preferably fewer than 3, preferably fewer than 2.

In one embodiment, to correct a deviation from an initial orthodontic treatment, the posterior part of the orthodontic treatment is defined such that at least some of the posterior orthodontic aligners are orthodontic aligners defined by the initial orthodontic treatment plan.

In particular, it may be defined such that corrective orthodontic aligners are inserted, at regular intervals or not, preferably evenly, between the orthodontic aligners defined by the initial orthodontic treatment plan. For example, a corrective orthodontic aligner may be inserted after each orthodontic aligner of the initial orthodontic treatment plan or every two or every 3 orthodontic aligners of the initial orthodontic treatment plan. The corrective orthodontic aligners are thus interposed between the orthodontic aligners designed and manufactured for the initial orthodontic treatment.

For example, if a deviation is detected specifically for tooth no 22 (or for a set of teeth, for example teeth no 22, 23 and 24), after use of orthodontic aligner no 3, the posterior part of the orthodontic treatment can define a first corrective orthodontic aligner similar to orthodontic aligner no 4 of the initial orthodontic treatment plan except for tooth no 22 (or for teeth no 22, 23 and 24). According to the posterior part of the orthodontic treatment plan, the user will therefore wear the first corrective orthodontic aligner, and then orthodontic aligner no 4. If the posterior part provides the interposition of two corrective orthodontic aligners, the posterior part of the orthodontic treatment can define a second corrective orthodontic aligner similar to orthodontic aligner no 5 of the initial orthodontic treatment plan except for tooth no 22 (or for teeth no 22, 23 and 24). According to the posterior part of the orthodontic treatment plan, the user will therefore wear the first corrective orthodontic aligner, then orthodontic aligner no 4, then the second corrective orthodontic aligner, and then orthodontic aligner no 5.

Irrespective of the embodiment, the series of corrective orthodontic aligners may in particular include one, two, three, four, five or six corrective orthodontic aligners.

Preferably, if the user has one or more orthodontic aligners referred to as “initial” that are intended for use, according to said initial orthodontic treatment plan, for a period covered by said posterior part of the orthodontic treatment, the user is informed that they must use the posterior orthodontic aligners instead of or in addition to said initial orthodontic aligners, before or interposed with the initial orthodontic aligners.

Preferably, each initial orthodontic aligner and each posterior orthodontic aligner preferably bear a mark, for example a number, for linking the posterior orthodontic aligner with the initial orthodontic aligner it is to replace.

More preferably still, in step C) and in general, said posterior part of the orthodontic treatment is defined such that the number of posterior orthodontic aligners is between 1 and 50, preferably fewer than 40, preferably fewer than 30, preferably fewer than 20, preferably fewer than 15, preferably fewer than 10. Preferably, the number of posterior orthodontic aligners is between 1 and 30, preferably between 1 and 15, preferably between 3 and 15, more preferably still between 5 and 15. The orthodontic treatment can thus be regularly adjusted and compliance is optimal.

In particular, the number of posterior orthodontic aligners is between 1 and 5, better is less than or equal to 3, preferably at least when the posterior part is a step of finishing the orthodontic treatment.

The posterior part may define a treatment which does not lead to an arrangement of the teeth according to the target model defined for the end of the orthodontic treatment. Since the number of orthodontic aligners is limited, the posterior part generally defines a treatment which does not lead to an arrangement of the teeth according to the target model defined for the end of the orthodontic treatment, unless the current instant is close to the end of the orthodontic treatment.

In one embodiment, the posterior part does not form part of the orthodontic treatment but is a support treatment, that is to say a post-orthodontic treatment step. The “posterior part” of the orthodontic treatment thus denotes any active or passive treatment phase after the anterior part of the orthodontic treatment.

In particular, the number of posterior orthodontic aligners may be 1 when the posterior part is a post-orthodontic treatment step, the orthodontic aligner being a support appliance. Within the context of a relapse, post-orthodontic treatment, the one or more posterior orthodontic aligners are determined so as to make it possible to return to a final dental configuration of the orthodontic treatment.

An orthodontic treatment can displace the teeth more or less rapidly, depending on the instant in question. When the rate of displacement is high, the risk of deviation from the orthodontic treatment, that is to say the risk that an orthodontic aligner is no longer suitable for the desired objective, increases. It is then preferable to follow the orthodontic treatment more precisely, and therefore preferable to give the user fewer orthodontic aligners. Preferably, in step C), the number of posterior orthodontic aligners is therefore determined as a function of the risk of a poor fit of the last posterior orthodontic aligner or as a function of a rate of change in shape of the dental arch during the posterior part of the orthodontic treatment.

The number of posterior orthodontic aligners may be determined as a function of the risk of a poor fit and/or a selection made by the dental healthcare professional and/or a constraint imposed by the orthodontic aligner manufacturer.

The number of posterior orthodontic aligners may be determined as a function of an analysis of the dental situation at the current instant, notably as a function of orthodontic indices. The orthodontic indices may be selected from: a Bolton index, an occlusion class, an overbite, an overjet. Preferably, the analysis of the dental situation includes analyzing orthodontic indices with regard to the user's biological data, for example the age, the state of a gum tissue, a bone density. This analysis can be performed on the basis of the current representation and/or the current model.

The risk of a poor fit can be evaluated statistically, that is to say by evaluating the probability that the desired objective will not be attained, by comparisons between “historical” situations experienced by other users, referred to as “historical users”, and the situation experienced by the user for whom the method according to the invention is implemented, or “current user”.

The variables for characterizing these situations can in particular comprise:
evaluations of the movements of one or more teeth (in rotation and/or in translation), in particular by means of movement predictions, for example by comparing movement predictions with real movements performed for the historical situations,
characteristics of the orthodontic appliances worn by the historical users and by the current user, for example relating to the use of cleats or the materials of the orthodontic aligners,
an evaluation of compliance,
characteristics relating to the orthodontic treatment, in particular
relating to the implementation of specific techniques, for example defining the presence or absence of stripping and/or expansion and/or distalization, and/or
relating to the detection of a deviation during the orthodontic treatment, for example unseating of the orthodontic aligner.

Preferably, the statistical evaluation of the risk of a poor fit takes into account at least the intended tooth movements, preferably as a function of the type of tooth, preferably as a function of the type of orthodontic appliance used to perform each movement.

The posterior part of the orthodontic treatment is linked to a set of elementary displacements for each tooth, the succession of these elementary displacements constituting a “pathway” of the tooth from its arrangement at the start of said posterior part to its arrangement at the end of said posterior part.

The statistical evaluation makes it possible to attribute, for each tooth, an elementary risk linked to each elementary displacement of a tooth, at a given instant and for a type of treatment, for example as a function of the nature of the orthodontic aligners. Preferably, the elementary risk is linked to the direction of the displacement (direction of a translational displacement or of an axis of rotation), preferably to the sense of the displacement (for example intrusion movement or opposing movement, rotation in the sense of the hands of a watch, or in the opposing sense), and preferably to the rate of said displacement.

For example, by analyzing the historical situations, it is possible to deduce that, with orthodontic aligners made of a particular polymer, an intrusion movement of 0.5 mm of an incisor, at a rate of 1 mm per month, from a given position of said incisor, is linked, in 60% of the historical situations, 3 weeks after said movement, to an unacceptable deviation of the orthodontic treatment from the orthodontic treatment plan. An elementary risk of 60% can then be attributed to such a movement for a future instant 3 weeks after the movement.

Upon each elementary displacement along a pathway of a tooth, it is thus possible to calculate an elementary risk for one and the same future instant, and therefore to link a risk with the pathway. For example, the first elementary displacement can lead to an elementary risk of 60% at the future instant, the second elementary displacement can lead to an elementary risk of 40% at the future instant, etc. The risk linked with the pathway up to the future instant can be for example the average of the elementary risks.

At a future instant, it is lastly possible to associate a risk of a poor fit, taking into account all of the risks over the pathways of all of the teeth, for example by averaging these risks. For example, the risk linked with the pathway of the incisor up to the future instant can be 50% and the risk linked with the pathway of a molar up to the future instant can be 30%. The risk of a poor fit for the future instant that is linked to the pathways of said incisor and said molar can be 40%.

A risk of a poor fit can thus be linked to each future instant.

In an advantageous embodiment, the duration of the posterior part and the number of orthodontic aligners are defined as a function of an acceptable threshold, or “poor-fit threshold”, for the risk of a poor fit. For example, the poor-fit threshold may be 50%. In the preceding example, the future instant is therefore too far in the future. Therefore, the risk of a poor fit is evaluated for one or more new future instants, increasingly close to the current instant, so as to reduce the risk of a poor fit until it is less than the poor-fit threshold.

In one embodiment, the risk of a poor fit is determined at least at future instants corresponding to intermediate instants at which it is intended that the user changes an orthodontic aligner. A risk of a poor fit is thus linked to each orthodontic aligner. The number of posterior orthodontic aligners can therefore be determined as a function of the risk of a poor fit of the last posterior orthodontic aligner, in particular by comparing the risk of a poor fit of the last posterior orthodontic aligner with the poor-fit threshold.

To determine the number of posterior orthodontic aligners, the method preferably comprises the following successive steps:
determining from the current representation, for example from a current model, a reference treatment plan adapted to all or some of the user's treatment from the current instant;
for each of a plurality of instants of the reference treatment plan, preferably at least for each intermediate instant corresponding to a change of orthodontic aligner intended in the reference treatment plan, attributing a probability of the risk of a poor fit, preferably as described above;
thresholding said probabilities of the risk of a poor fit so as to determine, from among said instants of the reference treatment plan, the first instant for which the probability of the risk of a poor fit exceeds a poor-fit threshold;
determining the posterior part, and therefore the number of posterior orthodontic aligners, such that it ends at the latest at said first instant.

For example, if the reference treatment plan intends the implementation of 30 orthodontic aligners, and if it is evaluated that the fourteenth and fifteenth orthodontic aligners carry the probability of the risk of a poor fit of 80% and 90%, respectively, for a poor-fit threshold fixed at 85%, the number of posterior orthodontic aligners may be 14, with the posterior part corresponding to the part of the treatment plan including orthodontic aligners 1 to 14.

Preferably, in step C), the number of orthodontic aligners to be manufactured is defined as a function of a rate of displacement of the teeth of the arch, preferably as a function of the rate of displacement of the teeth of the arch during the posterior part of the orthodontic treatment. The rate of displacement of the teeth of the arch may depend on the type of orthodontic aligner, for example the thickness of the orthodontic aligner, the shape of the orthodontic aligner, the material of the orthodontic aligner, the presence of cleats on the patient's teeth and/or prescriptions made by the orthodontist, for example how long the orthodontic aligner is worn each day, how often the orthodontic aligner is changed.

A neural network can be trained to determine the probability of poor fit of an orthodontic aligner.

In particular, a neural network can be trained on the basis of historical records relating to “historical” situations experienced, at “historical” instants, by historical users, each record comprising
a “historical” description of the historical situation, said historical description including at least one “historical” representation of the historical user's dental arch at the historical instant, and
“historical” information about the occurrence of a poor fit after an interval of time after the historical instant.

By providing the historical description as input and said historical information as output, the neural network learns to provide a probability for the occurrence of a poor fit after said interval of time after the current instant, if it is provided with a “current” description, corresponding to the historical descriptions, as input.

The current description “corresponds” to the historical descriptions in that it provides the same data, but for the “current” situation experienced by the user at the current instant, and not for the historical situations. In particular, the current description provides at least the current representation.

Preferably, it also provides data about the displacements of the teeth linked to the current situation, preferably data about the speeds of the teeth at the current instant.

Of course, the neural network may be designed to provide a risk of a poor fit for a tooth or for a set of teeth.

In one embodiment, the neural network is trained to directly provide a number of posterior orthodontic aligners.

In particular, the neural network can be trained on the basis of historical records relating to “historical” situations experienced, at “historical” instants, by historical users, each record comprising
a “historical” description of the historical situation, said historical description comprising at least “historical” representations of the historical user's dental arch at the historical instant and at a target instant after the historical instant, and a “historical” number of orthodontic aligners implemented to modify the arrangement of the historical user's teeth from the historical representation at the historical instant to the target historical representation;
“historical” information about the occurrence of a poor fit after an interval of time after the historical instant.

By providing the historical description as input and said historical information as output, the neural network learns to provide a probability for the occurrence of a poor fit after said interval of time after the current instant, if it is provided with a “current” description, corresponding to the historical descriptions, as input.

The current representation may be used to check whether the orthodontic treatment is progressing in accordance with the corresponding treatment plan. Preferably, after step A), “current” information is determined from the current representation, then
the current information is compared with anticipated theoretical information, at a simulation instant before the current instant, for the current instant, referred to as “anticipated information”, so as to define a compliance score, then
comparing the compliance score with a threshold value, and then,
depending on the result of said comparison,
steps B) and C) are carried out, or
orthodontic aligners in accordance with the treatment plan are manufactured, and said orthodontic aligners are presented to the user or to the orthodontist.

Advantageously, the posterior orthodontic aligners are therefore designed in step C) only if the orthodontic treatment is not considered to be progressing normally, that is to say in accordance with the treatment plan. The method can therefore be implemented more quickly. This also reduces the computing resources required.

The orthodontic aligners are considered to be “in accordance” with the treatment plan when they are defined by the treatment plan, with a view to carrying out the orthodontic treatment.

The simulation instant may be, for example, more than 1 week, 2 weeks, or 4 weeks before the current instant. The simulation instant may be in particular the instant at which the treatment plan linked to the orthodontic treatment was produced, generally immediately before the start of the orthodontic treatment or during a step B) of a previous cycle.

Preferably, the compliance score is defined:
by comparing the current model with a model of the orthodontic aligner intended to be worn by the user at the current instant, and/or
by comparing the current model with a theoretical model of the dental arch, produced at the simulation instant to represent the dental arch in its configuration at the current instant, and/or
by comparing the shapes of the orthodontic aligner and of the dental arch that are represented in the current representation.

Preferably, in step C), the posterior orthodontic aligners presented to the user are sent to them by post.

Preferably, the cycle of steps A) to C) is repeated more than once, more than twice, more than 3 times, and/or less than 7 times, less than 6 times during the orthodontic treatment. The duration of a cycle is preferably shorter than 1 month, 3 weeks, 2 weeks or 1 week and/or longer than 1 day.

Preferably, the orthodontic treatment is segmented into a plurality of orthodontic treatment parts, and the method comprises said cycles for each group of two consecutive said parts, said consecutive parts being, for a said cycle, said anterior and posterior parts of the orthodontic treatment.

Preferably, step B) and/or step C), preferably step B) and step C), are implemented by computer, by the computer alone or in collaboration with a dental healthcare professional, in particular an orthodontist.

The invention also relates to:
a computer program comprising program code instructions for implementing a method according to the invention, autonomously or in interaction with an operator, for example an orthodontist,
determining the treatment plan before step A),
drafting and/or sending reminder notifications before step A), and/or
determining the current model in step B),
designing the posterior orthodontic aligners in step C), and/or
determining the current information and/or anticipated information, updating the treatment plan at the start of step C), and/or
comparing the current and anticipated information,
   a computer-readable storage medium on which such a program is stored, for example a memory or a CD-ROM, and
   a computer into which such a program is loaded.

The computer program may in particular comprise program code instructions for, autonomously or in interaction with an operator, for example an orthodontist,
determining the treatment plan before step A),
drafting and/or sending reminder notifications before step A), and/or
determining the current model in step B),
designing the posterior orthodontic aligners in step C), and/or
determining the current information and/or anticipated information, updating the treatment plan at the start of step C), and/or
comparing the current and anticipated information.

The invention also relates to a system comprising:
preferably a first computer, optionally integrated in the cellphone, configured to send a reminder to the user to acquire said current representation using the cellphone;
the cellphone, configured to acquire said current representation;
a second computer, identical to or different than the first computer, preferably remote and communicating with the cellphone, capable of receiving said current representation from the cellphone and designing, on the basis of the current representation, the set of posterior orthodontic aligners, preferably capable of producing models of said posterior orthodontic aligners;
a machine for manufacturing orthodontic aligners, capable of manufacturing the set of posterior orthodontic aligners, preferably from models generated by said second computer;
preferably a transport network capable of transporting the set of posterior orthodontic aligners from said manufacturing machine to the user.

### Definitions

A “user” is understood to be any person for whom a method according to the invention is implemented, whether this person is ill or not.

An “orthodontic treatment” is all or part of a treatment intended to modify the configuration of a dental arch. An orthodontic treatment requires the use of one or more orthodontic appliances. An orthodontic treatment starts at an “initial” instant and ends at a “final” instant at which the intention is that the dental arch will have reached the targeted configuration.

An orthodontic treatment is planned using a “treatment plan”. A distinction is therefore made between the “orthodontic treatment”, which denotes a series of operations which take place in reality, and the “treatment plan”, which is the result of designing the orthodontic treatment. The treatment plan therefore comes before the orthodontic treatment.

An orthodontic treatment by means of orthodontic aligners is the practical application of a treatment plan which defines models for the dental arch in anticipated shapes, before the orthodontic treatment, for various instants during the orthodontic treatment. The generation of a treatment plan conventionally includes designing and modeling one or more orthodontic aligners. One example of designing aligners is described in “History of Orthodontics” by Basavaraj Subhashchandra Phulari. The treatment plan thus defines models for the implemented orthodontic aligners, these models being used to manufacture the corresponding orthodontic aligners. The orthodontic aligners may be modeled automatically, by computer, or manually by a dental healthcare professional.

More specifically, conventionally a series of models of the user's arch, which represent consecutive arch configurations, and a series of models of corresponding orthodontic aligners are determined, these series making it possible to manufacture orthodontic aligners each designed to modify the configuration of the arch from a configuration represented on an arch model to the configuration represented on the next arch model.

An orthodontic treatment, models of the arch at the start and at the end of the associated orthodontic treatment, and one or more orthodontic aligners designed to attain a configuration of the dental arch in accordance with the model of the dental arch at the end of the orthodontic treatment therefore “correspond” to each treatment plan.

An example of software for manipulating tooth models and creating a treatment plan is the Treat program, which is described on the webpage https://en.wikipedia.org/wiki/Clear_aligners#cite_note-invisalignsystem-10. U.S. Pat. No. 5,975,893A also describes the creation of a treatment plan.

An “orthodontic appliance” is an appliance which is worn or intended to be worn by a user. An orthodontic appliance may be intended for a therapeutic or prophylactic treatment, but also for an esthetic treatment. An orthodontic appliance may be in particular an orthodontic aligner. Such an aligner extends so as to follow the successive teeth of the arch to which it is attached. It defines a tray with the overall shape of a “U”. The configuration of an orthodontic appliance may be in particular determined to ensure its attachment to the teeth, but also as a function of a target positioning desired for the teeth. More specifically, the shape is determined such that, in the use position, the orthodontic appliance exerts stresses which tend to displace the treated teeth toward their target positioning (active orthodontic appliance) or to keep the teeth in this target positioning (passive orthodontic appliance, or “support” appliance).

A 3D scanner, or “scanner”, is an appliance for obtaining a model of a dental arch. A “portable scanner” weighs less than 1 kg, preferably less than 500 g and/or more than 50 g and the largest dimension of the portable scanner is less than 30 cm, 20 cm or 15 cm and/or greater than 5 cm. The portable scanner is configured to be manipulated by hand. It is preferably integrated in the cellphone.

The “use position” is the position of an orthodontic aligner when it has been attached to the arch in order to treat this arch. Conventionally, the attachment can be detached by the user by simply pulling on the aligner.

When an aligner is attached to an arch in the use position, teeth that do not sit correctly in the aligner (a situation referred to as “unseating”) and teeth that sit correctly in the aligner are referred to as “unseated teeth” and “non-unseated teeth”, respectively. Detection of “unseating” corresponds to the detection of unseated teeth. A dental healthcare professional knows full well how to distinguish between unseated teeth and non-unseated teeth. This distinction can also be made by a computer, in particular by evaluating the distance between a tooth and the bottom of the tray of the aligner that is attached thereto.

A configuration of an arch is referred to as a “real” configuration when it is the configuration that the user's arch has in reality. A configuration of an arch is referred to as a “theoretical” or “anticipated” configuration when it is a “simulated” or “intended” configuration of the patient's arch at a future instant. A theoretical configuration may be in particular produced by an orthodontist, preferably by displacing tooth models of a model of the dental arch that was produced before the start of the orthodontic treatment, for example using the Treat program.

A “model” is understood to mean a digital three-dimensional model. A model consists of a set of voxels.

A “tooth model” is a three-dimensional digital model of a tooth of the user's arch. A model of an arch may be segmented so as to define tooth models for at least some of the teeth and preferably all the teeth represented in the model of the arch. The tooth models are therefore models within the model of the arch. FIG. 7 shows an example of a view of an arch model segmented into tooth models 32. Computational tools for manipulating the tooth models of an arch model exist. These tools allow constraints to be set, in particular in order to limit the displacements of the tooth models to realistic displacements, for example in order to prevent adjacent tooth models from intersecting.

An arrangement of tooth models is therefore a model.

An “image” is understood to mean a two-dimensional image, such as a photograph or an image extracted from a film. An image is formed by pixels.

An “image of an arch”, “view of an arch”, “representation of an arch”, “scan of an arch”, or “model of an arch” is understood to mean an image, a view, a representation, a scan or a model of all or part of said dental arch, preferably representing at least 2, preferably at least 3, preferably at least 4 teeth. FIG. 6 shows an example of a view of an arch model.

“Metaheuristic” methods are known optimization methods. They are preferably chosen from the group formed by
evolutionary algorithms, preferably chosen from:
evolution strategies, genetic algorithms, differential-evolution algorithms, estimation of distribution algorithms, artificial immune systems, path relinking, shuffled complex evolution, simulated annealing, ant-colony-optimization algorithms, particle-swarm-optimization algorithms, taboo search, and the GRASP method;
the kangaroo algorithm, the Fletcher-Powell method, the noise method, stochastic tunneling, random-restart hill climbing, the cross-entropy method, and
hybrid methods combining the abovementioned metaheuristic methods.

Deep-learning algorithms are deep-learning devices that are well known to those skilled in the art. They comprise “neural networks” or “artificial neural networks”.

Those skilled in the art will be able to choose a neural network, depending on the task to be performed. Notably, a neural network may in particular be selected from:
networks specializing in image classification, called “CNN” (“convolutional neural network”), for example AlexNet (2012), ZF Net (2013), VGG Net (2014), GoogleNet (2015), Microsoft ResNet (2015), Caffe: BAIR Reference CaffeNet, BAIR AlexNet, Torch: VGG_CNN_S, VGG_CNN_M, VGG_CNN_M_2048, VGG_CNN_M_1024, VGG_CNN_M_128, VGG_CNN_F, VGG ILSVRC-2014 16-layer, VGG ILSVRC-2014 19-layer, Network-in-Network (Imagenet & CIFAR-10), Google: Inception (V3, V4),
networks specializing in locating and detecting objects in an image, object detection networks, for example R-CNN (2013), SSD (Single Shot MultiBox Detector: Object Detection network), Faster R-CNN (Faster Region-based Convolutional Network method: Object Detection network), Faster R-CNN (2015), SSD (2015), RCF (Richer Convolutional Features for Edge Detection) (2017), SPP-Net, 2014, OverFeat (Sermanet et al.), 2013, GoogleNet (Szegedy et al.), 2015,
VGGNet (Simonyan and Zisserman), 2014, R-CNN (Girshick et al.), 2014, Fast R-CNN (Girshick et al.), 2015, ResNet (He et al.), 2016, Faster R-CNN (Ren et al.), 2016, FPN (Lin et al.), 2016, YOLO (Redmon et al.), 2016, SSD (Liu et al.), 2016, ResNet v2 (He et al.), 2016, R-FCN (Dai et al.), 2016, ResNeXt (Lin et al.), 2017, DenseNet (Huang et al.), 2017, DPN (Chen et al.), 2017, YOLO9000 (Redmon and Farhadi), 2017, Hourglass (Newell et al.), 2016, MobileNet (Howard et al.), 2017,

DCN (Dai et al.), 2017, RetinaNet (Lin et al.), 2017, Mask R-CNN (He et al.), 2017, RefineDet (Zhang et al.), 2018, Cascade RCNN (Cai et al.), 2018, NASNet (Zoph et al.), 2019, CornerNet (Law and Deng), 2018, FSAF (Zhu et al.), 2019, SENet (Hu et al.), 2018, ExtremeNet (Zhou et al.), 2019,
NAS-FPN (Ghiasi et al.), 2019, Detnas (Chen et al.), 2019, FCOS (Tian et al.), 2019, CenterNet (Duan et al.), 2019, EfficientNet (Tan and Le), 2019, AlexNet (Krizhevsky et al.), 2012
networks specializing in image generation, for example Cycle-Consistent Adversarial Networks (2017), Augmented CycleGAN (2018), Deep Photo Style Transfer (2017), FastPhotoStyle (2018), pix2pix (2017), Style-Based Generator Architecture for GANs (2018), SRGAN (2018).

The above list is non-limiting.

Training a neural network consists in confronting it with a learning base containing information about the two types of object that the neural network must learn to “match”, that is to say connect to one another.

Training may be carried out on the basis of a learning base consisting of records each comprising a first object of a first type and a corresponding second object of a second type.

As an alternative, training may be carried out on the basis of a learning base consisting of records each comprising either a first object of a first type or a second object of a second type, each record however comprising information relating to the type of object that it contains. Such training techniques are for example described in the article by Zhu, Jun-Yan, et al. “Unpaired image-to-image translation using cycle-consistent adversarial networks.” Training the neural network with these records teaches it to provide, on the basis of any object of the first type, a corresponding object of the second type.

The quality of the analysis carried out by the neural network depends directly on the number of records in the learning base. Preferably, the learning base contains more than 10 000 records.

A measure of the difference between these two objects is referred to as a “match” or “fit” between two objects. A match or a fit is greatest (“best fit”) when it results from optimization that makes it possible to minimize said difference.

Two images, or “views”, that exhibit a best match substantially represent one and the same object in the same way. In other words, the representations of the object in these two images are able to be substantially superimposed.

A model exhibits a best match with an image when it has a view that exhibits a best match with said image.

The determination of the match may result from the comparison between two images. Preferably, this comparison itself results from the comparison of two corresponding maps. A measure of the difference between two maps or between two images is conventionally called a “distance”. A map may show discriminating information, which is characteristic information that may be extracted from an image (“image feature”), conventionally by computerized processing of this image. The discriminating information is preferably chosen from the group consisting of contour information, color information, density information, distance information, gloss information, saturation information, information on reflections and combinations of these forms of information. The discriminating information is preferably contour information.

“Comprise”, “include”, “have” or “exhibit” are to be interpreted broadly and non-limitingly, unless indicated otherwise.

### BRIEF DESCRIPTION OF THE DRAWINGS

Other features and advantages of the invention will become more clearly apparent on reading the following detailed description and on examining the appended drawing, in which:

FIG. 1 schematically illustrates a method according to the invention;

FIG. 2 shows one example of a dental retractor;

FIG. 3 schematically shows one example of a kit according to the invention;

FIG. 4 schematically shows the kit according to the invention in a use position, with the user being shown from the front;

FIG. 5 schematically shows the kit according to the invention in a use position, with the user being shown from the side;

FIG. 6 shows one example of a current model acquired using a portable scanner integrated in the cellphone and comprising 5000 points;

FIG. 7 shows one example of a model with segmented tooth models (only the tooth models have been shown);

FIG. 8 shows one example of a system according to the invention;

FIG. 9 illustrates one example of implementing the method according to the invention;

FIG. 10 schematically shows the determination of a number of posterior aligners for a method according to the invention.

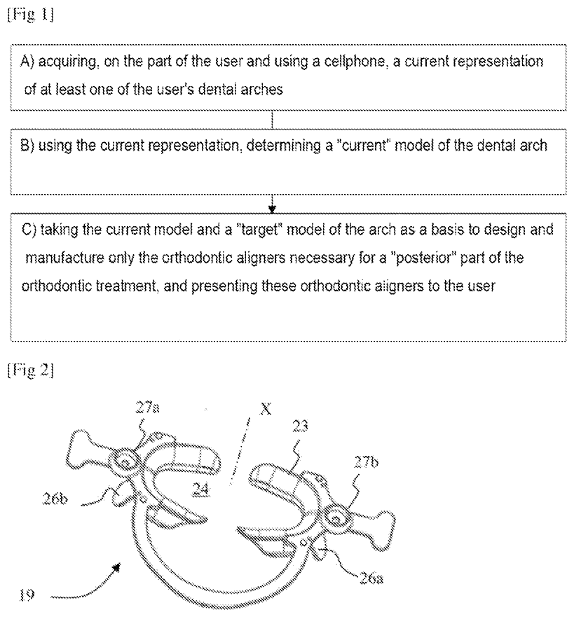

Other details and advantages of the invention are given in the following detailed description, which is given illustratively and non-limitingly.

### DETAILED DESCRIPTION

For a treatment by means of orthodontic aligners, a treatment plan for all of the orthodontic treatment is initially designed, conventionally by an orthodontist, in order to modify the configuration of the user's arch through to a “final” or “target” configuration.

The treatment plan leads to the determination of intermediate configurations for the arch that are anticipated, at the time when the treatment plan is developed, for various intermediate instants between the initial instant of the orthodontic treatment and the final instant at which the final configuration should be attained. Conventionally, the orthodontic aligners necessary to implement the treatment plan are then all designed according to the treatment plan and manufactured. Methods for designing the treatment plan and manufacturing the orthodontic aligners as a result are well known, for example the Clincheck software from Invisalign makes it possible to design treatment plans and the orthodontic aligners as a result.

All the orthodontic aligners are therefore conventionally designed and manufactured at the start of the orthodontic treatment. It is therefore necessary to subject computing resources and manufacturing machines to load for all the orthodontic aligners. As explained in the preamble, this loading is however partially needless, since the orthodontic treatment does not always progress as intended, that is to say according to the treatment plan.

The basis of the invention is the segmentation of the orthodontic treatment into multiple “parts” which follow one another from the start to the end of the orthodontic treatment, with the treatment plan being re-examined, and possibly updated, preferably shortly before each part.

A part of the orthodontic treatment makes it possible to change from one arch configuration to another, for example from the initial configuration to an intermediate configuration, for example the first intermediate configuration, or from one intermediate configuration to another, for example the next, or from an intermediate configuration, for example the last intermediate configuration, to the last configuration.

A part of the treatment plan and a set of orthodontic aligners comprising one or more orthodontic aligners are therefore linked to each orthodontic treatment part. The orthodontic aligners of a set are ordered depending on the period in which they are intended to be used. The “first” orthodontic aligner is therefore that which is to be used at the start of the orthodontic treatment part, the “last” orthodontic aligner is that which is to be used at the end of the orthodontic treatment part.

The orthodontic treatment may be segmented into more than 2, more than 5, more than 10, more than 20, more than 30 and/or less than 70 parts.

The duration of a part, preferably of any part, is preferably shorter than 20 weeks, preferably shorter than 15 weeks, preferably shorter than 10 weeks, preferably shorter than 8 weeks, preferably shorter than 6 weeks, preferably shorter than 5 weeks, or even shorter than 4 weeks, and preferably longer than 1 week or two weeks.

The durations of the parts preferably differ by less than 10 weeks, preferably less than 7 weeks, preferably less than 5 weeks, and preferably more than 1 week or two weeks. The duration of a part may be all the longer if the development of the dental situation, according to the treatment plan, is slow. However, the durations of the parts are preferably all substantially identical.

The number of orthodontic aligners in a set linked to an orthodontic treatment part, preferably in any set, may be identical or different depending on the part in question.

The number of orthodontic aligners in a said set is preferably fewer than 30, preferably fewer than 20, preferably fewer than 15, preferably fewer than 10, preferably fewer than 5, or even fewer than 3, or fewer than 2. Advantageously, the advantages afforded by the invention are optimized. Limiting the number of orthodontic aligners also limits the risk of losing an orthodontic aligner or inverting the use order of the orthodontic aligners.

Preferably, each part of an orthodontic treatment comprises a set of 1 to 15 orthodontic aligners, preferably a set of 3 to 15 orthodontic aligners, more preferably still a set of 5 to 15 orthodontic aligners. A step of finishing the orthodontic treatment may comprise 3 orthodontic aligners. Preferably, the last 10 parts of an orthodontic treatment are finishing steps, preferably the last 5 parts of an orthodontic treatment are finishing steps, preferably the last 3 parts of an orthodontic treatment are finishing steps. A post-orthodontic treatment step may comprise one orthodontic aligner, said orthodontic aligner being a support appliance.

In one embodiment, the numbers of orthodontic aligners of the sets of orthodontic aligners differ by less than 10, preferably less than 5, preferably are substantially identical, and the durations of the respective parts of the orthodontic treatment are variable.

In a preferred embodiment, the durations of all the parts of the orthodontic treatment differ by less than 3 weeks, preferably are substantially identical, and the number of orthodontic aligners of the sets of respective orthodontic aligners is variable. This embodiment has the advantage of accustoming the user to regularly and actively contributing to their orthodontic treatment by acquiring current representations.

In one embodiment, each part is linked to a set comprising a single orthodontic aligner and the duration of the part is the intended use duration for said aligner.

For at least one group of two successive parts, preferably for each group of two successive parts, a cycle of steps according to the invention is implemented.

Such a cycle of steps is described in the remainder of the description. For the sake of clarity, the two parts of the corresponding group are referred to as “anterior” and “posterior” parts, the associated treatment plans are referred to as “anterior” and “posterior” treatment plans, respectively, the associated sets of orthodontic aligners are referred to as a whole as “anterior” and “posterior”, respectively, and contain the “anterior” and “posterior” orthodontic aligners, respectively.

In step A), during an anterior part of the orthodontic treatment, at an instant referred to as “current” instant, the user or one of their relatives uses a cellphone to acquire a current representation of the dental arch receiving the orthodontic aligners, referred to as “current representation”.

The current instant is before or at the instant intended for the end of the anterior part of the orthodontic treatment, or “anterior final instant”. It is preferably determined such that the cycle can be completed before the anterior final instant, in order to not interrupt the orthodontic treatment. However, it is preferably determined to be as close as possible to the anterior final instant in order that the current representation is suitable to the best possible extent for determining the posterior orthodontic aligners. The temporal interval between the current instant and the anterior final instant is preferably longer than 1 week, and/or preferably shorter than 1 month, preferably shorter than three weeks, preferably shorter than two weeks.

In one embodiment, the current instant is after the instant intended for the end of the anterior part of the orthodontic treatment, or even after the end of the orthodontic treatment, in particular when the one or more orthodontic aligners to be manufactured are support appliances.

Preferably, a notification is sent to the user before the current instant to remind them it is necessary to acquire said current representation. Preferably, the notification indicates a date before which the user should acquire the current representation, this date preferably being less than 2 days around the date the notification was sent.

The notification may be in paper form or, preferably, electronic form, preferably sent to the cellphone, for example in the form of an email, an automatic alert from a specialized mobile application or an SMS. Such a reminder may be sent by the orthodontic practice or laboratory or by the dental healthcare professional or by a specialized application loaded on the cellphone.

The notification may be sent systematically. It is preferably conditional on the user not having already acquired said current representation.

Preferably, several of said notifications are sent in succession, until the user acknowledges receipt of the notification or has acquired the current representation requested.

### Retractor

To acquire the current representation using a cellphone, the user preferably uses a dental retractor 19.

The retractor 19 may have the characteristics of conventional retractors.

As shown in FIG. 2, it preferably comprises a rim 23 that extends around a retractor opening 24 of axis X and is arranged so that the user's lips can rest thereon while allowing the user's teeth to be seen through said retractor opening 24.

Preferably, the dental retractor comprises lugs 26a and 26b for moving the cheeks out of the way, so that the acquiring appliance, preferably a cellphone, can acquire, through the retractor opening, the vestibular surfaces of teeth disposed at the back of the mouth, such as molars.

### Mouthpiece

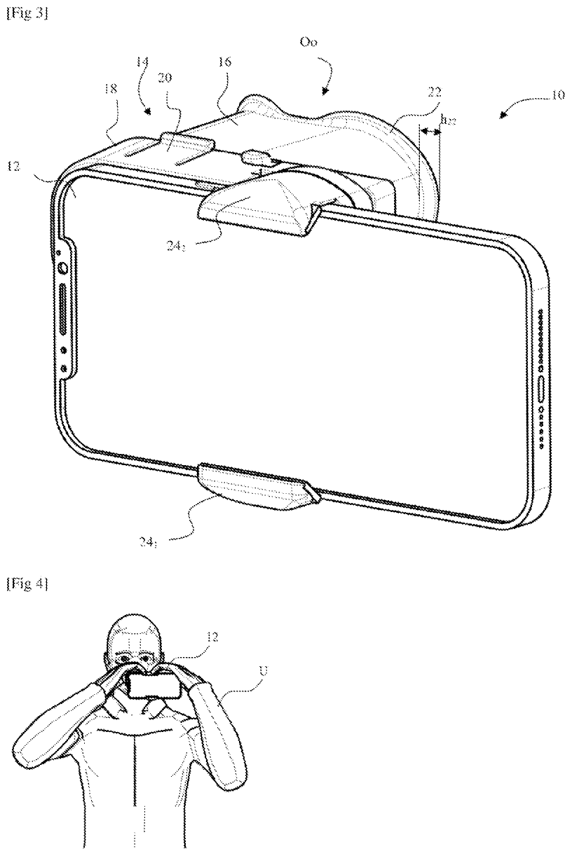

In a particularly advantageous embodiment illustrated in FIGS. 3 to 5, the user uses a kit 10 comprising the cellphone 12 and a support or “mouthpiece” 14 (FIG. 3) for, at the same time,
spreading the user's lips so as to reveal the teeth, and
making it easier to position and orient the cellphone 12 in relation to the teeth.

The mouthpiece 14 preferably takes the overall shape of a tubular body of which one opening, referred to as “oral opening” Oo, is intended to be introduced into the patient's mouth, and of which the opposite opening, referred to as “acquisition opening”, faces the lens of the camera of the cellphone, which is rigidly attached, preferably removably, to the mouthpiece 14.

Preferably, the acquisition opening also faces a flash of the cellphone, thereby making it possible to use the flash to illuminate the user's teeth during the acquisition.

The mouthpiece 14 makes it possible to define a spacing between the cellphone and the oral opening Oo and also an orientation of the cellphone with respect to the oral opening. Advantageously, in the use position, the data acquired by the cellphone 12 through its lens, the acquisition opening and the oral opening are thus acquired at a given distance from the user's teeth. Preferably, the mouthpiece 14 is configured such that the spacing between the oral opening and the cellphone and the orientation of the cellphone around the axis of the oral opening are predefined and constant.

Preferably, the mouthpiece 14 comprises:
a tubular retractor 16 which defines the oral opening Oo and preferably comprises a rim 22 extending radially outward, around the periphery of the oral opening Oo, that is intended to be introduced between the user's lips and teeth, and
an adapter 18 to which the cellphone 12 is attached, for example clamped between two jaws 241 and 242, as illustrated in FIG. 3, the adapter 18 being attached rigidly to the tubular retractor 16, preferably removably, for example by means of a clip 20, or being made in one piece with the retractor, such that the lens of the cellphone can “see” the oral opening.

The maximum height h22 of the rim 22 is preferably greater than 3 mm and less than 10 mm.

To acquire the current representation, the user joins the tubular retractor 16 to the adapter 18 by means of the clip 20, and then joins the cellphone to the adapter 18 such that the cellphone can acquire the current representation through the tubular retractor 16 and the adapter 18. They then introduce that end of the tubular retractor that is opposite the cellphone into their mouth by inserting the rim 22 between their lips and the teeth. The lips thus rest on the tubular retractor 16, on the outside of the latter, this making it possible to clearly view the teeth through the oral opening Oo.

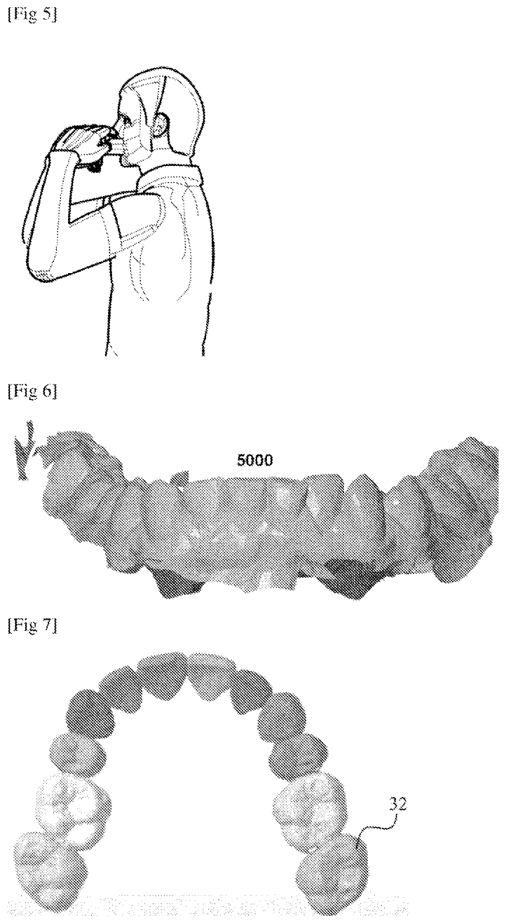

In the use position obtained, illustrated in FIGS. 4 and 5, the teeth do not rest on the mouthpiece, such that the user U may, by turning the head in relation to the mouthpiece, modify the teeth that are visible to the cellphone through the oral opening. They may also modify the distance between their dental arches. In particular, the mouthpiece moves the lips apart but does not bear against the teeth so as to move the two jaws apart.

The current representation may represent all or some of a dental arch or of the two dental arches.

The rim may be replaced by a dental retractor 19. The dental retractor 19 and the cellphone are preferably attached removably to the mouthpiece.

The dental retractor may be attached to the mouthpiece by one or more fasteners 27a and 27b, for example magnetic fasteners (FIG. 2).

Preferably, a specialized application is loaded in the cellphone to guide the user, preferably orally and/or visually, through the various operations to be carried out to acquire the current representation.

The current representation is preferably extraoral, that is to say acquired without the cellphone entering the user's mouth.

Preferably, the current representation is acquired “with bare teeth”, that is to say while no orthodontic aligner is attached to the user's teeth.

The current representation may be made up of one or, preferably, more images, referred to as “current images”.

Preferably, the user acquires at least 3, better at least 5, even better at least 10 current images. They may notably acquire a front view, a view from the left, a view from the right and/or an occlusal view. They may acquire at least one view with the jaws open and/or at least one view with the jaws closed.

### Evaluation of the Suitability of the Orthodontic Treatment Followed to the Treatment Plan

In one embodiment, the degree to which the last anterior orthodontic aligner fits the real configuration of the user's dental arch is evaluated at the current instant and, if this degree of fit is unsatisfactory, the treatment plan is updated.

Preferably, the suitability of the last anterior orthodontic aligner to the orthodontic treatment is checked by computer, preferably on the basis of the current representation. In particular, “current” information can be extracted from the current representation and then compared with corresponding anticipated information (that is to say relating to the same variable as the current information), at a simulation instant, for the current instant, referred to as “anticipated information”.

The current information may be simple, for example a distance between two teeth, a height of a tooth from the gum, or an orientation of a tooth, or be complex, for example be a model providing the shape of one or more teeth of the dental arch.

The simulation instant is for example the instant at which the treatment plan was developed before the start of the orthodontic treatment or, preferably, the instant of the last update to the treatment plan.

The simulation instant may be more than 1 week, more than 2 weeks, more than 3 weeks, or more than 4 weeks before the current instant.

For example, according to the treatment plan, it was anticipated that, at the current instant, the spacing between the two incisors would be 0.2 mm. The current information shows that this spacing is 0.5 mm.

The suitability of the last anterior orthodontic aligner to the orthodontic treatment can also be evaluated by examining the difference in shape between the last anterior orthodontic aligner, used at the current instant, and the shape of the dental arch at the current instant, preferably by comparing the current model and a model of the last anterior orthodontic aligner.

The suitability of the last anterior orthodontic aligner to the orthodontic treatment can be evaluated by examining the difference in shape between the shape of the dental arch at the current instant and the shape of the dental arch in a theoretical model of the dental arch representing the dental arch in its configuration at the current instant, preferably by comparing the current model and a theoretical model of the dental arch produced at the simulation instant to represent the dental arch in its configuration at the current instant.

The suitability of the last anterior orthodontic aligner to the anterior orthodontic treatment can also be evaluated by evaluating the unseating of the last anterior orthodontic aligner,
by analyzing one or more current images representing it in the use position and/or
by comparing the current model with the model of said last anterior orthodontic aligner, and/or
by comparing one or more current images with the model of said last anterior orthodontic aligner and/or the theoretical model of the dental arch used to design said last anterior orthodontic aligner.

For this last embodiment, it is possible in particular to search for a view of said last anterior orthodontic aligner which best fits the current image, and then compare this view and this current image.

Preferably, a neural network is trained to evaluate the unseating of the last anterior orthodontic aligner. The neural network may be for example trained by providing it with pairs formed by a current model and an orthodontic aligner model as input and an evaluation of the unseating of this orthodontic aligner when it is worn by the dental arch modeled by the current model as output.

To determine unseating, generally the contour of the teeth and the contour of the bottom of the tray are compared, the presence of a difference making it possible to detect the unseating of the aligner. Specifically, the bottom of the orthodontic tray, if there is no unseating, has a substantially complementary shape to that of the free ends of the teeth.

The comparison may consist in finding the difference between the current and anticipated information, said difference defining a compliance score representative of the suitability of the treatment plan to the reality observed.

In one embodiment, the compliance score makes it possible to decide whether the treatment plan should be modified. In particular, if the compliance score is compared to a threshold value, and the performance of steps B) and C) can be conditional on the result of the comparison. For example, if the compliance score is a measurement of the degree of unseating of the orthodontic aligner, it is possible to decide that steps B) and C) will only be performed if this measurement is less than a threshold value, for example less than 0.5 mm.

The compliance score may also take into account the detection of particular events, for example the loss of a cleat and/or one or more deviations noted at the current instants for anteriorly implementing the method according to the invention. For example, the compliance score can be reduced, or even be zero, if a loss of a cleat is detected or if two successive deviations, in particular instances of unseating, have already been noted.

In one embodiment, steps B) and C) are systematically implemented and the degree to which the last anterior orthodontic aligner fits the real configuration of the user's dental arch is not evaluated. This is because such an evaluation is not indispensable, since the posterior treatment plan is calculated at the current instant, independently of this evaluation. Advantageously, the computing resources used are limited as a result. Moreover, this embodiment makes it possible to manufacture posterior orthodontic aligners which take account of the real configuration of the dental arch at the current instant, that is to say at the moment when the first of these posterior orthodontic aligners is to be used. This therefore improves the effectiveness of the orthodontic treatment.

In step B), a current model can then be generated from these current images.

The current model may be generated on the cellphone, for example by means of a specialized application loaded on the cellphone. As an alternative, the current images are sent, for example by means of the cellphone, to a remote computer, said computer being configured to generate a current model from the current images.

The computer can receive the current images from multiple users.

Preferably, each current image is a photograph or is an image extracted from a film. The current images are preferably in color, preferably in real colors. More preferably still, they are photographs showing a real dental arch as it would be perceived by the human eye, unlike a tomographic image or a panoramic image acquired by X-ray.

Generation of the Current Model from Current Images

The current model may be generated from current images by means of a neural network, said neural network being trained to generate a model representing said dental arch from images of dental arches.

Preferably, the following steps are carried out in order to train a neural network:
I. creating a learning base comprising more than 1000, preferably more than 5000, preferably more than 10 000, preferably more than 30 000, preferably more than 50 000, preferably more than 100 000 historical records, each comprising a historical set of dental arch images and a historical model, with each image, referred to as “historical image”, being a best fit with a view of the historical model;
II. training at least one neural network, by means of the learning base, by providing it, for each record, with the historical set of images as input and the historical model as output.

Once it is trained, a set of current images is submitted to the neural network such that it determines a model representing the dental arch shown in the current images at the current instant.

As an alternative, the neural network is trained to generate a current model from a library of tooth models and current images.

Preferably, for each current image, the following steps are carried out:
a) identifying the zones of the current image that represent a tooth and the number of said tooth, preferably by means of a neural network;
b) for each of said zones,
searching a historical library comprising more than 1000, preferably more than 5000, preferably more than 10 000, preferably more than 30 000, preferably more than 50 000, preferably more than 100 000 records, each record comprising a tooth model, referred to as “historical tooth model”, and a corresponding tooth number,
preferably by means of a neural network, for a historical tooth model
having a tooth number identical to that of the tooth represented by said zone and
being the closest match to said tooth represented by said zone in said current image, or “optimum tooth model”;
c) arranging the set of optimum tooth models so as to create a current model which is the best fit with the current images.

The neural network used in step a) to identify the tooth zones can be trained by providing it with images representing dental arches as input and the zones representing the teeth in said images as output.

The neural network used in step a) to determine the tooth numbers can be trained by providing it with tooth zones as input and the numbers of the teeth represented in these zones as output.

The neural network used in step b) can be trained by providing it with historical tooth models as input and the numbers of the teeth modeled by the historical tooth models as output.

The tooth number may be replaced with another tooth attribute, for example a type of tooth, a shape parameter of the tooth, for example a tooth width, in particular a mesio-palatal width, a thickness, a crown height, an index of mesial and distal deflection of the incisal margin, or a level of wear, an appearance parameter of the tooth, in particular a translucence index or a color parameter, a parameter relating to the state of the tooth, for example “worn”, “broken”, “decayed” or “fitted” (that is to say in contact with a dental appliance, preferably orthodontic appliance), an age for the user, or a combination of these attributes. A tooth attribute is preferably an attribute which relates only to the tooth modeled by the tooth model.

As an alternative, the following steps are carried out:
i. before the current instant, preferably before the start of the orthodontic treatment, acquiring a three-dimensional model of the dental arch, or “initial model”, by means of a 3D scanner, preferably on the part of a dental healthcare professional;
ii. segmenting the initial model to define tooth models;
iii. at the current instant, taking current images as a basis to displace and/or deform the tooth models of the initial model, for example by optimization by means of a metaheuristic method, in particular selected from the methods provided above, or by means of a neural network, so as to generate a current model which exhibits the best match with the current images.

The current representation may be made up of a model constituting the current model.

In a preferred embodiment, the current model is acquired by means of a portable scanner. The portable scanner may advantageously be loaded in the cellphone. Preferably, the portable scanner is a LIDAR, for “light detection and ranging”.

The portable scanner may have low accuracy and serve as a basis for reconstructing a more accurate model. Specifically, all that is needed is to take note of the spatial position of several notable points of the arch and then search for the tooth models which can be disposed in a configuration compatible with the position of these points. Advantageously, the generation of a low-accuracy model is possible with limited technical means, which can be made movable. A low-accuracy model also requires little memory in order to be stored.

In step C), the current model and a target model of the dental arch are taken as a basis to design, manufacture and then send to the user solely the posterior orthodontic aligners.

The target model may be an intermediate model determined while the treatment plan for the orthodontic treatment was being determined. The dental configuration modeled by the target model then corresponds to an expected dental configuration at an instant marking a change in orthodontic aligner, according to the treatment plan defined before the start of the orthodontic treatment.

The target model may be a “final” model of the dental arch, that is to say theoretically representing the dental arch at the end of the orthodontic treatment.

The treatment plan is therefore updated, at the start of step C), with the current model, thereby making it possible to design the posterior orthodontic aligners. The updating of the treatment plan can be performed in the same way as the initial generation of the treatment plan, before step A).

The initial generation of the treatment plan is conventionally based on an “initial” model of the dental arch, that is to say representing the dental arch before the orthodontic treatment, and on the “final” model of the dental arch, such that the orthodontic treatment makes it possible to modify the corresponding dental arch.

Similarly, the updating of the treatment plan is based on the current model and on the target model, such that the orthodontic treatment makes it possible to modify the corresponding dental arch. In other words, the treatment plan is redefined, but by replacing the initial model with the current model, and possibly by replacing the final model with a target model. The treatment plan thus makes it possible to design orthodontic aligners that are well suited to the real configuration of the dental arch at the current instant.

### Presentation of the Posterior Orthodontic Aligners

Preferably, the posterior orthodontic aligners are sent by post directly to the user. As an alternative, they may be sent to a dental healthcare professional, in particular the orthodontist, who then sends them to the user.

Preferably, instructions are sent to the user with the posterior orthodontic aligners that inform them, for example, about how long to wear each orthodontic aligner, and/or possible instants when the orthodontic aligners will be changed, and/or whether an appointment needs to be made with the orthodontist.

These instructions can be sent with the aligner, on the specialized cellphone application, by SMS, by email and/or by post.

### System

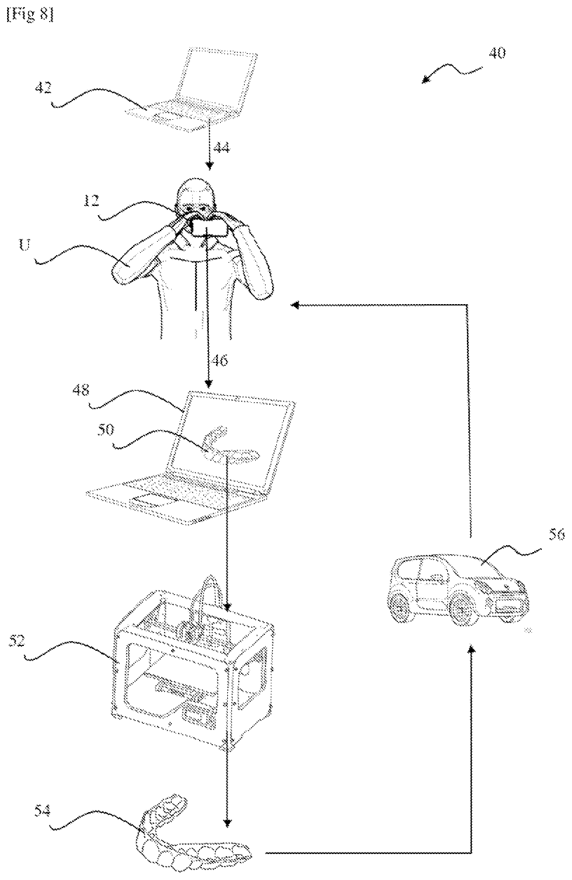

FIG. 8 illustrates a system 40 according to the invention.

This system 40 comprises:
preferably a first computer 42, configured to send a reminder 44 to the user U to acquire said current representation 46 using the cellphone 12;
the cellphone 12, configured to acquire said current representation 46;
a second computer 48, identical to or different than the first computer 42, optionally integrated in the cellphone 12, preferably remote and communicating with the cellphone 12, capable of receiving said current representation 46 from the cellphone and designing, on the basis of the current representation, the set of posterior orthodontic aligners, preferably capable of producing models 50 of said posterior orthodontic aligners;
a machine 52 for manufacturing orthodontic aligners, capable of manufacturing the set of posterior orthodontic aligners 54, preferably from models generated by said second computer;
preferably a transport network 56 capable of transporting the set of posterior orthodontic aligners from said manufacturing machine to the user U.

The first computer is preferably configured to send reminders to more than 100, more than 1000, or more than 10 000 users. The second computer is preferably configured to receive and process current representations from more than 100, more than 1000, or more than 10 000 users.

### Example

In a particular embodiment of the invention, the orthodontist designs, at an initial instant, a treatment plan for all the orthodontic treatment, and then at least some, preferably all of the orthodontic aligners needed to implement the treatment plan are all designed according to the treatment plan, manufactured and presented to the user.

As described above, the treatment plan is preferably segmented into several steps separated by intermediate instants. Each intermediate instant thus marks an instant at which it is intended for the user to change their orthodontic aligner.

Preferably, an intermediate model determining the expected configuration of the user's teeth is generated for each intermediate instant, preferably by displacing tooth models of an initial model representing the dental arch before the start of the orthodontic treatment.

Before an intermediate instant, preferably before each intermediate instant, preferably less than 10 days, less than 5 days or less than 2 days before said intermediate instant, compliance with the treatment plan is checked.

The check comprises evaluating the degree to which the last anterior orthodontic aligner fits the real configuration of the dental arch of the user. Preferably, it comprises acquiring a current representation of the dental arch, according to step A), preferably producing a current model, according to step B), and comparing this current representation, preferably the current model, to the intermediate model linked to the intermediate instant.

If this degree of fit is insufficient, one or more orthodontic aligners are designed and manufactured according to step C), the posterior part being determined such that at least some of the initially defined treatment plan can be implemented after implementing the treatment plan for the posterior part. In other words, said posterior part of the orthodontic treatment is defined, using a number of posterior orthodontic aligners of between 1 and 15, so as to correct the poor fit and catch up with the initial orthodontic treatment plan.

As a result, it is not necessary to redefine all the treatment plan from the current instant to the final instant. Moreover, once the posterior part of the orthodontic treatment is completed, the user can advantageously resume the orthodontic treatment as initially intended and use the corresponding orthodontic aligners that they have already received and that, according to the initial objective technical problem, should be used after said posterior part of the orthodontic treatment. Advantageously, only some of the orthodontic aligners initially manufactured will not be used.

Preferably, the posterior part covers a period of use of fewer than 5 orthodontic aligners, preferably a single orthodontic aligner.

Preferably, the posterior part is determined such that the teeth reach a configuration as provided in an intermediate model defined beforehand, preferably in an intermediate model corresponding to the intermediate model for the first intermediate instant immediately following the intermediate instant following the current instant.

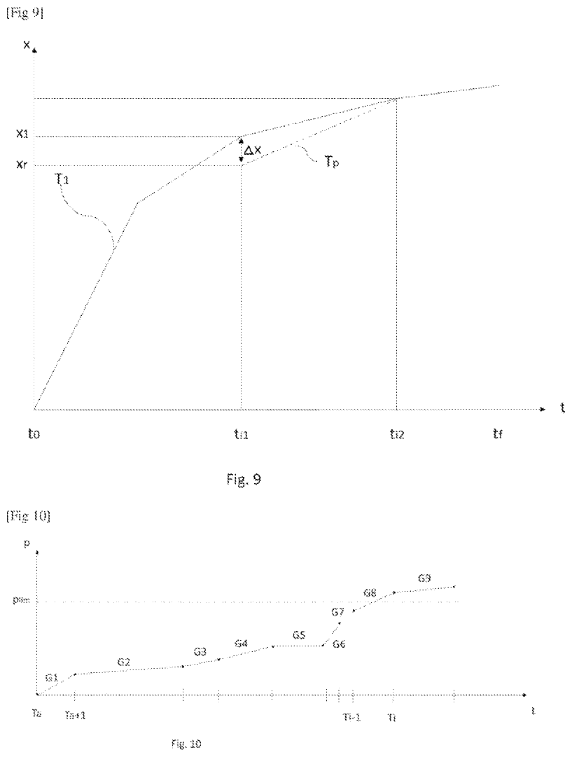

One example is illustrated in FIG. 9, in which x represents a variable at least partially defining the positioning of a user's tooth and t represents the time. The curve T₁ represents the treatment plan determined at the initial instant t₀, the treatment plan T₁ being determined such that the user's teeth reach a target configuration of the teeth at the final instant tr.

If, at an intermediate instant tᵢ₁ of the treatment plan T₁, an insufficient fit is detected, for example a difference Δx between the expected positioning of the tooth, x₁, and the real positioning of the tooth, xᵣ, is detected, one or more orthodontic aligners are designed and manufactured for the implementation of a posterior part Tₚ, the implementation of this posterior part making it possible to return to the initially intended treatment plan T₁, for example to return to the treatment plan at the following intermediate instant tᵢ₂. As a result, from the following intermediate instant tᵢ₂ the user can resume the orthodontic treatment as initially intended.

### Another Example

At a current instant, a treatment plan is determined from the current representation and/or a current model, the treatment plan being defined by one orthodontic aligner or a succession of orthodontic aligners and the instants at which these orthodontic aligners are changed.

A treatment plan is shown schematically in FIG. 10, comprising nine successive orthodontic aligners G1, G2, G3, G4, G5, G6, G7, G8 and G9.

In FIG. 10, the abscissa represents the instants at which the orthodontic aligner is changed, the orthodontic aligner G1 being for example provided between the instants Tₐ et Tₐ₊₁, and the ordinate represents the probability of the risk of an overall poor fit during the treatment. The probability of the risk of an overall poor fit is determined as a function of the movements of the teeth, the type of tooth to be shifted, the orthodontic aligner. Moreover, the probability of the risk of a poor fit can be determined as a function of the intended or performed movements of the teeth. Specifically, a tooth that has already performed significant movements will not develop in the same way as a tooth which has remained fixed in place. This probability is preferably determined by statistical analysis of historical situations.

As specified above, it is observed specifically that during the implementation of an orthodontic treatment it is not rare for one or more teeth to deviate from the initially intended treatment plan. By analyzing historical user situations, it is possible to determine the probability of the risks linked to each tooth, as a function of parameters such as the rate of rotation, the rate of movement in translation, the type of tooth.

Preferably, the probability of the risk of an overall poor fit is determined at an instant t by combining the probabilities of the risk of a poor fit of each of the teeth in the user's dental arch at this instant t.

As a result, it is possible to estimate a probability of the risk of a poor fit, that is to say a probability that the real configuration of the teeth after implementing the treatment deviates from the intended treatment plan, each time an orthodontic aligner is changed.

This estimation makes it possible to determine an optimum number of orthodontic aligners for the posterior part of the orthodontic treatment.

In the example of FIG. 10, the probability of the risk of a poor fit exceeds a threshold pₗᵢₘ between the instants Tᵢ₋₁ and Tᵢ, corresponding to wearing the aligners G8. In the present case, to avoid manufacturing possibly needless orthodontic aligners and to avoid a deviation of the orthodontic treatment, the number of orthodontic aligners of the posterior part is preferably selected between 1 and 7.

As is presently clear, a method according to the invention makes it possible to manufacture a minimum number of orthodontic aligners without the user needing to visit an orthodontist. The orthodontic treatment does not deviate, since the orthodontic aligners are still well suited to the real configuration of the dental arch. It is corrected regularly, thereby limiting the risk of the user needing to urgently visit the orthodontist.

Of course, the invention is not limited to the embodiments described above and shown. An orthodontic treatment may be therapeutic and/or esthetic.

The user is not necessarily a human being. In particular, a method according to the invention may be used for another animal.

1. A method for distributing orthodontic aligners intended for an orthodontic treatment of a user's dental arch, said orthodontic treatment being defined by an initial orthodontic treatment plan, said method comprising a cycle of the following steps:
A) at a current instant during an “anterior” part of the orthodontic treatment, acquiring, on the part of the user and using a cellphone, a current representation of said dental arch, then
C) updating the initial orthodontic treatment plan on the basis of the current representation, and defining, on the basis of the updated initial orthodontic treatment plan, a “posterior” part of the initial orthodontic treatment following said anterior part of the orthodontic treatment, and then
designing and manufacturing only the orthodontic aligners necessary for said “posterior” part, referred to as “posterior orthodontic aligners”, the number of posterior orthodontic aligners being more than or equal to 1 and fewer than 20, and
presenting the one or more posterior orthodontic aligners to the user.

2. The method as claimed in claim 1, comprising, between steps A) and C), the following step:
B) using the current representation, determining a digital three-dimensional model representing the dental arch at the current instant, referred to as “current” model,
in which method, in step C), said current model is used for said design and manufacture.

3. The method as claimed in claim 2, wherein, in step C), said current model is compared with a “target” digital three-dimensional model of the dental arch.

4. The method as claimed in claim 1, wherein, before the current instant, a notification is sent to said cellphone to provide a reminder of the need to acquire said current representation.

5. The method as claimed in claim 1, wherein,
in step A), the current instant is before the instant intended for the end of the anterior part of the orthodontic treatment, or “anterior final instant”, the temporal interval between the current instant and the first final instant being longer than 5 days and shorter than 1 month.

6. The method as claimed in claim 1, wherein, in step A),
a dental retractor or
a mouthpiece to which the cellphone is attached
is used by the user.

7. The method as claimed in claim 2, wherein,
in step A), the current representation is made up of a model acquired by a scanner integrated in the cellphone and/or of one or more images acquired by a camera integrated in the cellphone, and wherein,
in step B), the current model is made up of said acquired model or is generated from the current images and/or the acquired model.

8. The method as claimed in claim 2, wherein, in step B, the current model is generated;
by means of a portable scanner integrated in the cellphone, or
by means of a neural network from one or more current images acquired using the cellphone, or
by assembling “historical” tooth models as a function of a model acquired using a portable scanner integrated in the cellphone and/or one or more current images acquired using the cellphone, or
by deforming, by means of a metaheuristic method, a model of the dental arch generated before the current instant, such that the model deformed in this way best fits the one or more current images.

9. The method as claimed in claim 1, wherein, if before step C) a difference is observed between the position of the teeth at the current instant and a theoretical position of said teeth which is in accordance with said initial orthodontic treatment plan and is intended at the current instant, said posterior part of the orthodontic treatment is defined such that, during said posterior part of the orthodontic treatment, said teeth return to a position which is in accordance with said orthodontic treatment plan.

10. The method as claimed in claim 9, wherein the posterior part of the orthodontic treatment is defined to be inserted in the initial orthodontic treatment plan, such that all the orthodontic aligners defined by the initial orthodontic treatment plan are made use of according to the orthodontic treatment plan resulting from said updating, it being possible for the posterior part of the orthodontic treatment to be inserted in the initial orthodontic treatment plan in a block or be cut up into segments, the segments of the posterior part of the orthodontic treatment being inserted discontinuously in the initial orthodontic treatment plan.

11. The method as claimed in claim 10, wherein the number of posterior orthodontic aligners is fewer than 4.

12. The method as claimed in claim 10, wherein a cycle of steps A) to C) is implemented after the user has used a given number of orthodontic aligners.

13. The method as claimed in claim 1, wherein, in step C), if the user has one or more orthodontic aligners referred to as “initial” that are intended for use, according to said orthodontic treatment plan, for a period covered by said posterior part of the orthodontic treatment, the user is informed that they must use the posterior orthodontic aligners instead of or in addition to said initial orthodontic aligners.

14. The method as claimed in claim 1, wherein, in step C), said posterior part of the orthodontic treatment is defined such that the number of posterior orthodontic aligners is between 1 and 10, the posterior part of the orthodontic treatment being a step of finishing the orthodontic treatment.

15. The method as claimed in claim 1, wherein, in step C), said posterior part of the orthodontic treatment is defined such that the number of posterior orthodontic aligners is 1, the posterior part of the orthodontic treatment comprising or being a support step.

16. The method as claimed in claim 1, wherein,
in step C), the number of posterior orthodontic aligners is determined as a function of the risk of a poor fit of the last posterior orthodontic aligner.

17. The method as claimed in claim 1, wherein,
in step C), the number of orthodontic aligners to be manufactured is defined as a function of a rate of displacement of the teeth of the arch.

18. The method as claimed in claim 1, wherein the posterior orthodontic aligners (54) presented to the user are sent to them by post.

19. The method as claimed in claim 1, comprising between 1 and 7 cycles of steps A) to C).

20. A method for distributing orthodontic aligners intended for an orthodontic treatment of a user's dental arch, said method comprising a cycle of the following steps:
A) at a current instant during an “anterior” part of the orthodontic treatment, acquiring, on the part of the user and using a cellphone, a current representation of said dental arch, then
after step A), determining “current” information from the current representation, then
comparing the current information with anticipated information, at a simulation instant more than 1 week before the current instant, for the current instant, referred to as “anticipated information”, so as to define a compliance score, then
comparing the compliance score with a threshold value, and then,
depending on the result of said comparison of the compliance score with the threshold value,
manufacturing orthodontic aligners in accordance with a treatment plan defined at the simulation instant, and presenting said orthodontic aligners to the user or performing the following steps B) and C):
B) using the current representation, determining a digital three-dimensional model representing the dental arch at the current instant, referred to as “current” model, and then
C) taking the current model and a “target” digital three-dimensional model of the dental arch as a basis to design and manufacture only the orthodontic aligners necessary for a “posterior” part of the orthodontic treatment following said anterior part of the orthodontic treatment, referred to as “posterior orthodontic aligners”, and presenting the one or more posterior orthodontic aligners to the user.

21. The method as claimed in claim 20, wherein the compliance score is defined:
by comparing the current model with a model of the orthodontic aligner intended to be worn by the user at the current instant, and/or
by comparing the current model with a theoretical model of the dental arch, produced at the simulation instant to represent the dental arch in its configuration at the current instant, and/or
by comparing the shapes of the orthodontic aligner and of the dental arch that are represented in the current representation.

22. A system for implementing a method as claimed in claim 1, comprising:
preferably a first computer, configured to send a reminder to the user to acquire said current representation using the cellphone;
the cellphone, configured to acquire said current representation;
a second computer, identical to or different than the first computer, which is capable of receiving said current representation from the cellphone and producing, from the current representation, said posterior orthodontic aligners;
a machine for manufacturing orthodontic aligners, capable of manufacturing the posterior set of posterior orthodontic aligners from models generated by said second computer.

23. The system for implementing a method as claimed in claim 20, comprising a transport network capable of transporting the posterior set of orthodontic aligners from said manufacturing machine to the user.
